(19) United States
(12) Patent Application Publication
(10) Pub. No.: US 2017/0172526 A1
(43) Pub. Date: Jun. 22, 2017

(54) X-RAY DETECTOR SYSTEM

(71) Applicant: Teratech Corporation, Burlington, MA (US)

(72) Inventor: Alice Chiang, Burlington, MA (US)

(21) Appl. No.: 15/337,692

(22) Filed: Oct. 28, 2016

Related U.S. Application Data
(60) Provisional application No. 62/259,967, filed on Nov. 25, 2015.
(60) Provisional application No. 62/248,197, filed on Oct. 29, 2015.

(51) Int. Cl. A61B 6/00; G01T 1/20; A61B 6/03

(52) U.S. Cl. CPC A61B 6/4233; A61B 6/032; A61B 6/502; G01T 1/20; A61B 6/40; H01J 35/065

(57) ABSTRACT

The present invention relates to x-ray detector systems and methods for imaging of objects. The detector system can include a scintillator mounted to a solid state pixelated detector having a buried channel structure. An electronic shutter can be used to control detector exposure during an imaging procedure.

### CROSS REFERENCE TO RELATED APPLICATION

This is application claims priority to U.S. Provisional Application No. 62/259,967 filed on Nov. 25, 2015 and U.S. Provisional Application No. 62/248,197 filed Oct. 29, 2015, the entire contents of the above applications being incorporated herein by reference.

### BACKGROUND OF THE INVENTION

Digital x-ray imaging systems are now used for a wide variety of clinical applications including cardiology mammography interventional fluoroscopy procedures and many other known techniques. For medical applications, there is a continuing need to limit x-ray exposure by reducing the x-ray transmission flux required for these applications. A Computed Tomography (CT) scanning system can be used for many applications including imaging of the human anatomy in a medical imaging system as well as for baggage/container images in a security/inspection systems. To form one frame of a CT image of a patient, for example, a scanner acquires roughly 1000 sequential x-ray exposures, each with 0.5 ms to 1 ms of exposure time. The x-ray transmission flux is measured at each of those 1000 exposures. The processed transmission flux measurements are then used to reconstruct an image which reveals the anatomical structures in a slice taken through the patient. There are artifacts associated with the detector's delay response from the current exposure and the decay responses of previous exposures, which leads to blurring of the acquired image. For helical scanning in which the object being scanned is moved, there are additional artifacts associated with motion of the object which leads to blurring in an axial direction.

Thus, a continuing need exists for improvements in x-ray imaging and particularly for CT scanning systems.

### SUMMARY OF THE INVENTION

While there are many facets to the overall practice of using x-ray systems such as mammography, fluoroscopy, CT systems, etc., there are limited improvements in the technical features of those systems over the past decade relating to radiation dosage effects, spatial resolution and frame rate. For example, the mean glandular dose used in a typical 2D mammogram examination is above 1 mGy, and the detective quantum efficiency (DQE) of current systems is less than 20% at 8 lp/mm. Preferred embodiments of the present invention include a buried channel, low noise detector array, such as CCD, as an x-ray detector system. Improvements in the technical features of this system include lower electronic (capacitor reset or kTC) noise, lower glandular dose, finer spatial resolution, and faster frame rates than current systems.

In particular, preferred embodiments utilize a back-side illuminated buried channel charge coupled device (CCD) detector with a built-in electronic shutter that allows real-time dynamic imaging can provide these features. The system can have minimum image blur while the system is in read-out mode. The system can include on a wafer-scale, a three-side buttable buried channel CCD with an active area at least 10 cm×10 cm, with 4000-row and 4000-column pixels, for example, and a pixel size less than 800 square microns and preferably less than 625 square microns (25 um×25 um). The system can employ a digital time integration approach to improve the dynamic range. Furthermore, the system uses a solid state x-ray source and a flexible substrate for mounting of the detector array such that the array is curved along an arc that corresponds to an arc of substantially even intensity distribution for the emitted x-ray beam.

The present invention can further relate to x-ray imaging systems in which the detector system output is sampled at a rate to reduce motion artifacts. Digital integration with detector rise-and-fall time correction is used to reduce or eliminate image blurring associated with multiple sequential x-ray exposures.

An x-ray source emits x-ray radiation in a sequence of pulses at a selected exposure rate and detector output. A sampling circuit is used to sample the detector output at a rate higher than the x-ray exposure rate. In a preferred embodiment, analog-to-digital (A/D) converters can be used in sampling of the detector output signals. The A/D output clock rate is greater than the image exposure rate which enables correction based upon the detector's detected rise and fall characteristics. The detector elements in a given row can be multiplexed in the detector circuit. The detector can also be a direct x-ray detector in which the semiconductor is doped, for example, with lithium.

### BRIEF DESCRIPTION OF THE DRAWINGS

FIG. 1 is a schematic view of a CT scanner system is accordance with the invention.

FIGS. 2A and 2B show different angular projections of an object being scanned.

FIG. 3 shows a schematic view of a detector array having I rows and J columns of elements.

FIGS. 4A-4B are schematic graphical representations of a step function input waveform and a detected response, respectively.

FIGS. 5A and 5B illustrate a preferred embodiment of a data acquisition system in accordance with the invention.

FIG. 6A illustrates measured detector response of the first to the k^{th} view and the sub-sampling rate.

FIG. 6B illustrates a process sequence for acquiring a two or three dimensional image of an object such as an animal or human body in accordance with the invention.

FIG. 7 illustrates a transistor array for a preferred embodiment of the invention.

FIG. 8 illustrates an array of CCD output shift registers, each associated with the detector and data acquisition system.

FIG. 9 illustrates a timing diagram for an x-ray source and detector read-out.

FIGS. 10A and 10B illustrate a depletion region of a device storage well for an electronic shutter.

FIG. 11 illustrates a planar detector assembly for a back illuminated device with a scintillator coupled to the detector array.

FIG. 12 illustrates a single panel detector and a detector assembly formed by four abutted detector arrays.

FIG. 13 illustrates a process for fabricating a buried channel CMOS process for fabricating a detector in accordance with the invention.

FIG. 14 illustrates an N-channel implant and resulting electronic field for a detector in accordance with the invention.

FIGS. 15A-C illustrate a schematic representation of a device, a charge distribution, and an energy potential according to various embodiments of the present invention.

FIG. 16 illustrates an overview of a double-poly process for CCD/CMOS design according to various embodiments of the present invention.

FIGS. 17A-C schematically illustrate steps of a two-poly CCD/CMOS process in accordance with the invention.

FIG. 18 illustrates an overview of a single-poly process for CCD/CMOS design according to various embodiments of the present invention.

FIGS. 19A-C schematically illustrate steps of a one-poly CCD/CMOS process in accordance with the invention.

FIG. 20 depicts a method of imaging a region of interest within a patient with a buried channel detector device.

### DETAILED DESCRIPTION OF THE INVENTION

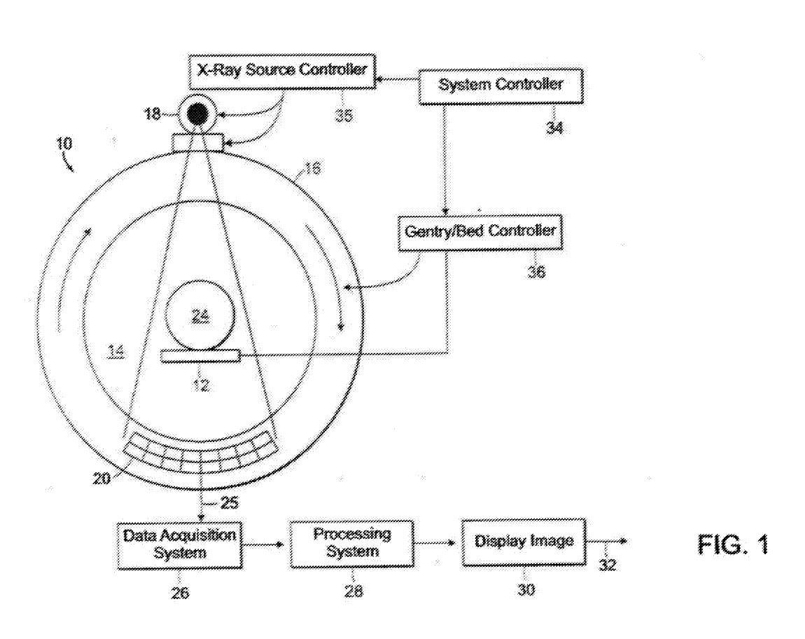

As can be seen in FIG. 1, a CT scanner 10 includes an object table or support 12 which is positioned within the center opening aperture 14 of a frame 16 or gantry. An x-ray source 18 is mounted within the gantry 16 to one side of the opening aperture 14, and a detector array 10 is mounted to the second side of the aperture 14. During scanning, the x-ray source and the detector array are rotated around the object 24. CT relies on the measurement of attenuated x-ray transmission flux through the object from different rotation angles to form an image. The x-ray flux after attenuation by an object impinging on the x-ray detector is measured and recorded using a data acquisition system 26, data processing system 28 and display 30. The images can then be transmitted 32 via wired or wireless connection to data storage or a network. A system controller 34 is connected to the x-ray source controller 35 and the gantry and support controller 36. The data acquisition system includes a sampling circuit device that samples the detector output signal 25 at a rate higher than the x-ray exposure rate. A preferred embodiment uses an analog-to-digital (A/D) converter that digitizes the detector output signals at a high resolution and speed.

Each attenuated measurement represents the summation or line integral of the attenuation coefficients of an object along a particular rotation angle or a ray path. Each set of measurements is referred to as a “view” or a “projection”, and the measurement data of the complete set is referred to as a transmission profile. Typically, a 360 degree gantry rotation is used to acquire a complete transmission profile. During the 360 degree rotation, a typical CT scanner acquires roughly 1000 views, corresponding to 1000 different angular orientations, i.e., a single frame or a single slice of CT image comprises roughly 1000 attenuated x-ray measurements. Each measurement corresponds to a particular angular orientation of the x-ray source and the detector array with an x-ray exposure time in a range of 0.1 to 5 millisecond and preferably of 0.5 to 1 millisecond (ms).

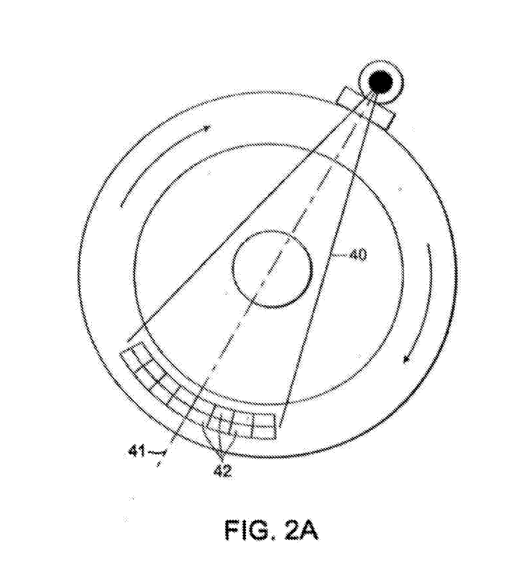

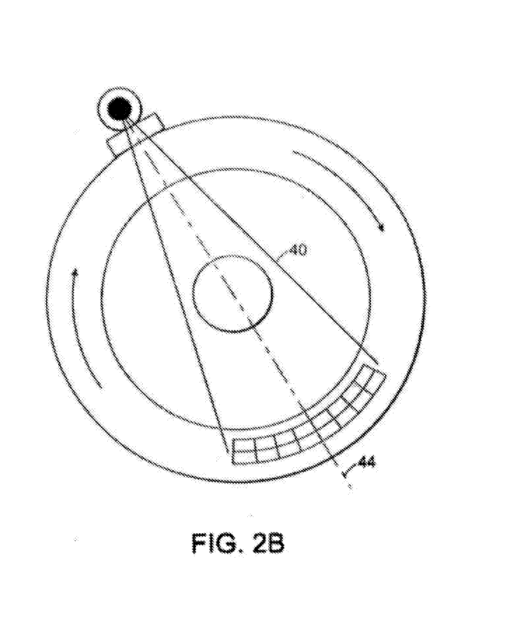

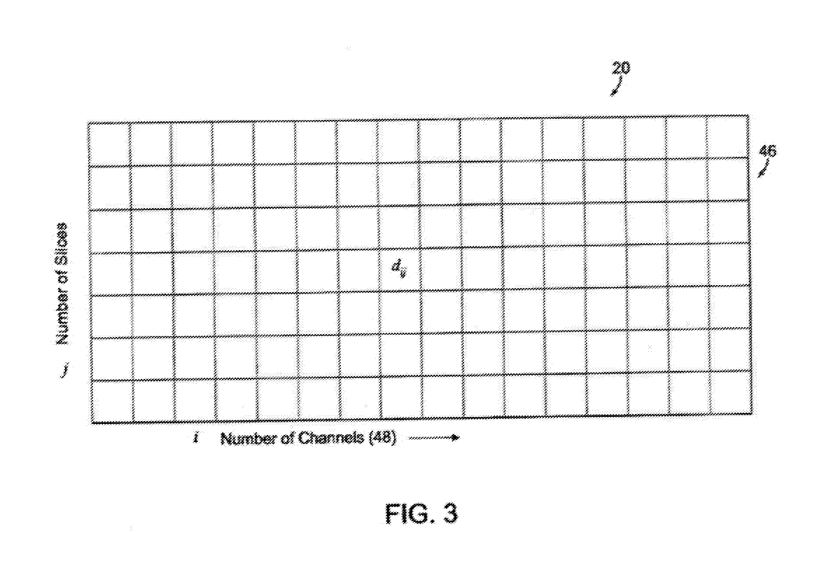

As shown in FIGS. 2A and 2B, the x-ray source produces a fan-shaped beam 40 that passes through the object and is received by an array of detector elements 42. Each detector element 42 in this array produces a separate attenuation signal and the signals from all the detector elements produce the transmission profile for the indicated angular orientation. FIG. 2A reflects a fan beam directed along one axis 41 and FIG. 2B shows the fan beam directed along a second axis 44. The transmission profiles from all different angular orientations are then used to reconstruct an image which reveals the anatomical structures in a slice taken through the object. A typical scanner has 800 to 1000 detector elements or channels 42 along a row to provide fine resolution. A CT image generated by a single row of the detector array is referred to as a “slice”. Conventionally, a CT scanner with a single row of detector array is referred to as a single slice CT, while a CT scanner that includes multiple rows 46 of such detector arrays is referred to as multi-slice CT, MSCT, or spiral CT. The number of slices corresponds to the number of rows of detector arrays. The MSCT was introduced in the early 1990s to offer the benefit of simultaneous acquisition of multiple slices of images of the patient and allow the acquisition of volume data without the danger of misregistration or double registration of anatomical details. Recent generations of MSCT systems acquire more than 64 slices per rotation. As can be seen in FIG. 3, a detector array includes i rows and j columns A single pixel in the detector array is represented by dᵢⱼ where i represents the detector channel number in a given row and j stands for slice number. In a medical CT scanner, typical element numbers of channels are 800 to 1000, typical slice numbers are 2, 4, 8, 16, 64, 256, etc.

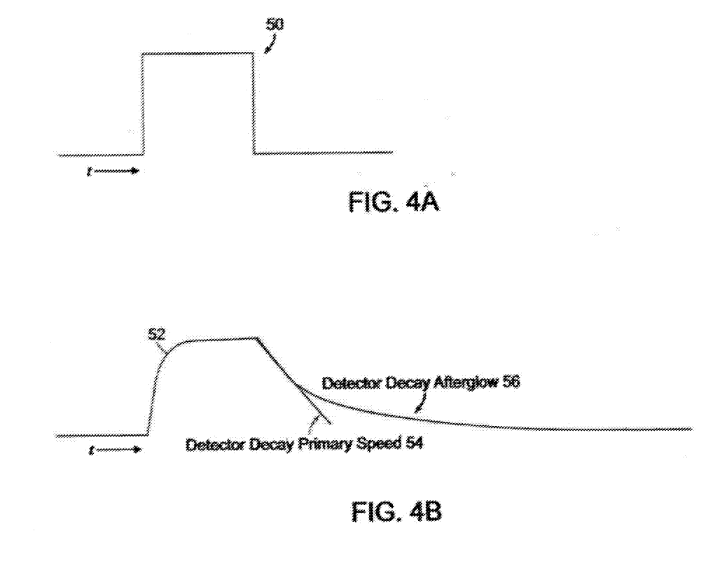

An x-ray detector can either be a photon counter or a solid state detector as described in greater detail below. The solid state detector offers the advantage of large packing density and is now most commonly used in all commercial CT scanners. Each solid state x-ray detector generally includes a scintillator and a solid state photodiode, or a solid state two dimensional array such as a CCD (Charge Coupled Device). Direct x-ray detectors can also be used for certain applications. The scintillator converts the incoming x-ray photons into optical photons. When an x-ray impinges on a scintillator, the optical photons are not emitted by the scintillator instantaneously; rather the emission follows a long decay curve. Furthermore, when the impinging x-ray is shut off, the emission of photons are not terminated instantaneously; instead it has a long decay time. The slow rise-and-fall time of a detector is shown in FIG. 4B in response to an x-ray with a step function input waveform 50 as shown in FIG. 4A. The detected response shown in FIG. 4B is characterized by a slow rise time 52 and a long decay time. The time dependence of the absorbed x-ray and the emitted photon intensity can be modeled as exponentials with different decay constants. In CT terminology, the decay time includes a primary decay factor 54 and afterglow factor 56. The primary decay factor 54 is the initial decay time constant. The remaining time constants are referred to as “afterglow”.

While measured x-ray transmission values can in principle be corrected arithmetically with slow (exponential) decay behavior, existing systems do not correct for the primary decay factor and initial afterglow less than 0.5 ms. This results in poorer dynamic performance and higher computer costs associated with image processing. A preferred embodiment of the invention utilizes a digital sampling system that corrects for effects due to both the primary speed and total afterglow. Additionally, in helical scanning mode where the object being scanned moves in an axial direction that is orthogonal to the plane of rotation of the source and detector while x-rays are being detected, motion artifacts can be created that can be addressed by the present invention. A preferred embodiment corrects each sub-sampled detector output before it is summed to provide the transmission profile. Thus, the present system corrects for overall cross channel blurring during helical scanning

Typically in existing CT systems the read-out of each detector element occurs at the end of each total x-ray exposure at each selected angle of rotation. Thus the sampling time is about the same as the exposure time, i.e., about 0.5 ms to 1.0 ms. At the end of each exposure, the total integrated electrons are sampled and read-out. In the present invention, each detector can be exposed to the same x-ray transmission pulse sequence and the same total exposure time as existing systems; however, the read-out is preferably at a much higher sampling rate. The output is converted to a digital representation and corrected for artifacts that occur during each detection interval. The measured transmission profile at each view (angle) represents more exactly the attenuated x-ray transmission. 2D and/or 3D image reconstruction can be carried out on the measured data to improve image quality and also reduce the x-ray exposure rate to increase patient safety.

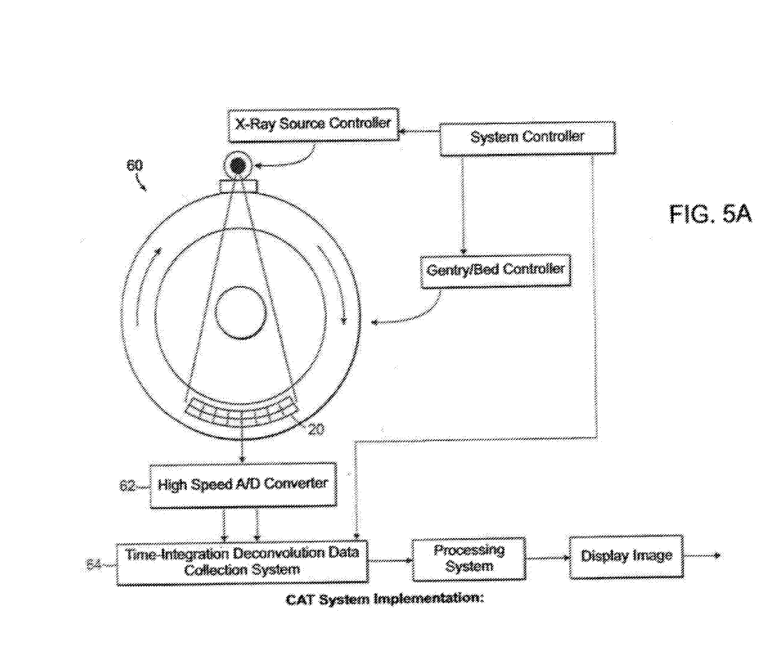

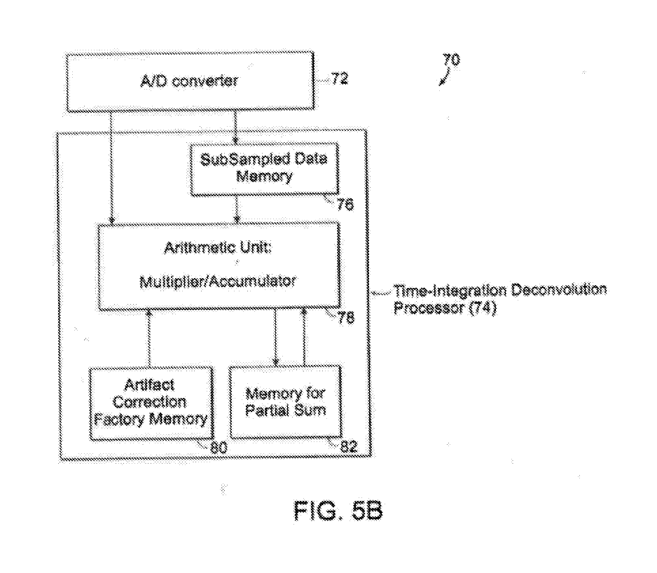

FIG. 5A illustrates a preferred embodiment of the invention in which the imaging system 60 as previously described has an AID converter 62 receiving output signals from the detector 20. Typically a 16-22 bit A/D is used where the converter operates at 1 kHz or more, preferably at 2 kHz or more. The output of the converter 62 is transmitted to a time integration deconvolution data collection system 64. An example of such a system 70 is shown in FIG. 5B. The output of converter 72 is transmitted to a sub-sampled data memory 76 and an arithmetic unit 78 of processor 74. The processor 74 also includes an artifact correction factor memory 80 and a memory 82 for partial summation.

In this example for a 64 slice by 1000 element detector array with a 0.5 second rotation rate collecting 1000 views each having 128 samples for each rotation, the AID conversion rate is 128×64×2k=16 MHz. If a more moderate 14 bit AID converter is used, the bit accuracy is 21 bits. The partial sum indicated in Eq. (6) is stored in memory 82. Memory 80 is used to store the detector artifact correction factors from Eq. 6. The sum generated by arithmetic unit 78 is the attenuated transmission corrected for primary speed and after glow.

The detector rise time response or the time dependence of the detector absorbed x-ray intensity can be modeled as

R(t)=aₙ(1−e^{-t/γ}^{ₙ}) for tₙ₋₁≦t<tₙ (1)

where aₙ represents the relative strength of the scintillator's x-ray-photon-to-optical-photon response component with time constant τₙ and n is determined from measurements of the detector rise curve for a given incoming x-ray flux. For example, a scintillator's x-ray response with three time constants can be modeled with such as

R(t)=a₁(1−e^{-t/γ}^{₁}) for 0≦t<t₁ (2)

a₂(1−e^{-t/γ}^{₂}) for t₁≦t<t₂

a₃(1−e^{-t/γ}^{₃}) for t₂≦t<t₃

The slope of a detector response at a given time t, R(t), is a unique function

$$\frac{d}{dt} R ( t ) = \frac{{a}_{n}}{{τ}_{n}} {e}^{- t \text{/} {γ}_{n}}$$

In particular, the initial slope \dot{R}(0) can be expressed as

$$\dot{R} ( 0 ) = \frac{{a}_{1}}{{τ}_{1}} \qquad ( 3 )$$

The time dependence of the detector emitted light intensity can be modeled as follows,

F(t)=bₘe^{-t/τ}^{ₘ} for tₘ₋₁≦t<tₘ (4)

where aₘ represents the relative strength of the detector decay component with time constant τₘ and M is determined from measurements of the detector decay curve. For example, it was reported in Kacheriess et al, “Advanced Single-Slice rebinning in cone beam Spiral CT,” Med. Phys. 27, 754-772 (2000), the entire contents of which is incorporated herein by reference,

F(t)=b₁e^{-t/τ}^{₁} for 0≦t<t₁ (5)

b₂e^{-t/τ}^{₂} for t₁≦t<t₂

b₃e^{-t/τ}^{₃} for t₂≦t<t₃

b₄e^{-t/τ}^{₄} for t₃≦t<t₄ (5)

where τ₁˜1 ms, τ₂˜6 ms, τ₃˜40 ms, and τ₄˜100 ms. Newer scintillation crystals having microsecond decay time with afterglow less than 0.1% of signal after 3 ms have been reported.

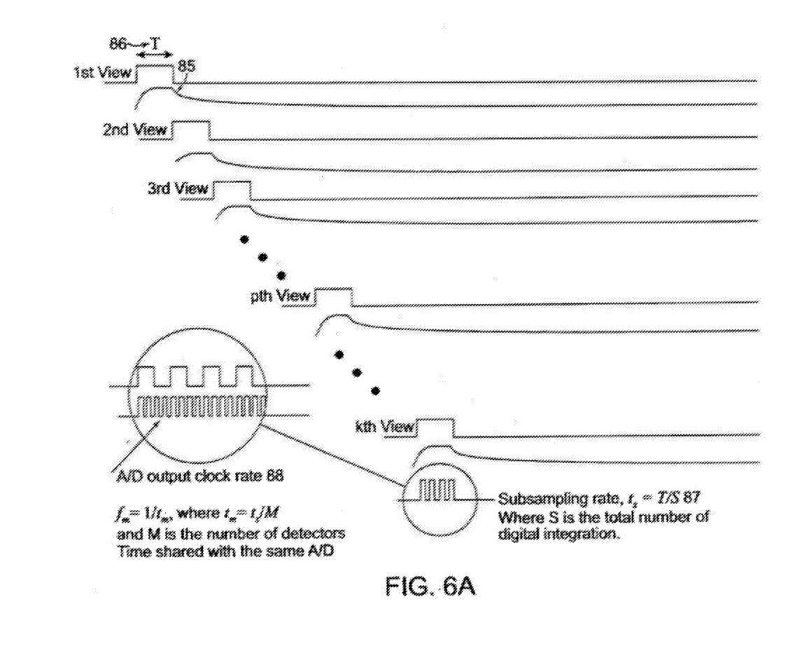

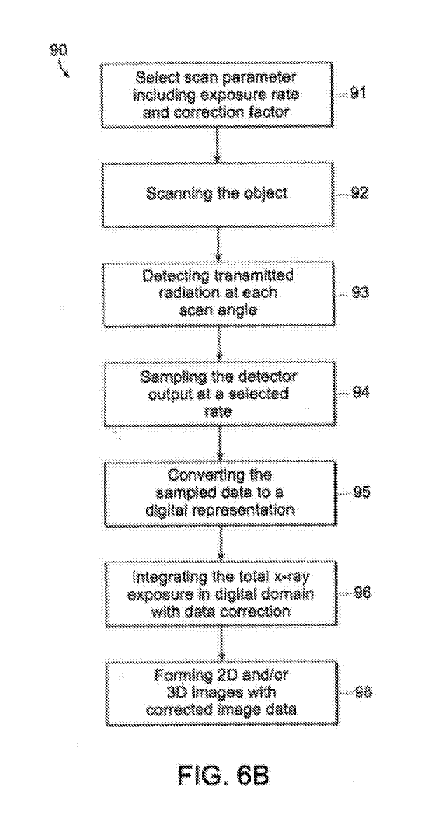

As the detector array rapidly rotates about the patient, the exponential decay blurs together detector readings for successive views. As shown in FIG. 6A, the measured detector response at k^{th} view, includes the detector response 85 due to the k^{th} x-ray exposure and also all (k-1) previous x-ray exposures each at interval 86. Those exponential decay blurring terms, due to the response time lag of the detector, is referred to as the “primary speed” term and the “afterglow” term, wherein the “primary speed” refers to the primary decay component of the detector and the “afterglow” refers to all the remaining components. The primary speed of the detector degrades the spatial resolution of the system, and the afterglow term degrades the azimuthal component of the image resolution. As shown above, the primary speed of a typical commercially used x-ray detector is about 1 ms. As stated above, a typical CT system acquires roughly 1000 views over a 360° rotation, the sampling time of each view is chosen to be about the same as the primary speed decay time constant of the detector. In currently available CT systems, corrective algorithms have been reported to compensate for the detector afterglow decay characteristic but not to correct for the primary speed term. The present invention provides an x-ray sampling system and method that allows correction for both “primary speed” term, the “afterglow” term of the emitted-light decay time constant and also provides for correction for the detector slow rise time of the absorbed x-ray.

Instead of using the detector to integrate the total x-ray exposures during each view, the present invention measures the detector outputs using a much higher sub-sampling rate 87 within each exposure, digitizing the higher sub-sampling rate samples, correcting the samples based on the detector rise-and-fall characteristics stored in memory 80 and then digitally integrating the corrected samples for the total exposure time of each view. For example, for a CT scanner with a 0.5 s rotation and 1000 views, the system utilizes a 0.5 ms exposure time or a 2 Khz sampling rate at each view. In this invention, an A/D converter is used to sample the detector outputs at a sub-sampling rate 88 of 64 kHz, preferably 128 kHz or more, or at a 7.8 μs intervals, i.e., a total of 64 samples are collected during each view. Each collected digital sample will be compensated for its detector decay time constants based on Equations (1) to (5).

It can be seen from FIG. 6A the detector output at the k^{th} view is the sum of all the sub-samplings during this period with the detector outputs properly compensated for both the detector rise time of the k^{th} view, also for the decay responses from all the previous k-1 views. Let T represents CT's sampling time for each view, or the exposure time at each view, and let S be the total sub-sampling number of this invention. Define tₛ=T/S, where tₛ is the sub-sampling time of the CT system and s is the running index representing the sub-sampling time. As described above and shown in the process of FIG. 6B, for example, for a given CT scan parameter are selected 91, the x-ray exposure time or the sampling time of each view is 0.5 ms, let us take 64 subsamples during this exposure time, it follows than tₛ=0.5 ms/64=7.8 us, or the sub-sampling rate is 128 KHz. The operation principle is as follows.
1. At any sub-sampling time of the first viewing angle, k=1, the transmit-attenuated detector input signal impinging at detector located at the i^{th} column and r^{th} row, xᵢⱼ(stₛ) is the measured detector output, yᵢⱼ(stₛ), compensated by its rise time.

xᵢⱼ(stₛ)=yᵢⱼ(stₛ)/a(1−e^{-st}^{ₛ}^{/γ}^{₁})

where γ₁ is the detector initial rise time constant.
2.At any sub-sampling time of the second 2^{nd} viewing angle, k=2, the detector input signal, xᵢⱼ(T+stₛ) is the sum of the measured output yᵢⱼ(T+stₛ) compensated by the detector's rise-time response and the decayed output due to the input x-ray impinging at the detector at first viewing angle, xᵢⱼ(1)

xᵢⱼ(T+stₛ)=yᵢⱼ(T+stₛ)/a(1−e^{-st}^{ₛ}^{/γ}^{₁})−xᵢⱼ(stₛ)b₁e^{-st}^{ₛ}^{/τ}^{₁}

where τ₁ is the detector primer decay constant.
3. At any sub-sampling time of the 3^{rd} view, the detector input signal, xᵢⱼ(2T+stₛ) is the sum of the measured output, yᵢⱼ(2T+stₛ) compensated by the detector's rise time response, the decayed output due to the input x-ray impinging at the detector at first viewing angle, xᵢⱼ(T) and the decayed output due to the input x-ray impinging at the detector at second viewing angle, xᵢⱼ(2T).

xᵢⱼ(2T+stₛ)=yᵢⱼ(2T+stₛ)/a(1−e^{-st}^{ₛ}^{/γ}^{₁})−xᵢⱼ(T+stₛ)b₁e^{-st}^{ₛ}^{/τ1}

−xᵢⱼ(stₛ)b₂e^{-(T+st}^{ₛ}^{)/τ}^{₂}

where τ₂ is the decay time constant for T≦t<2T.
4. At any sub-sampling time of the k^{th} view, the detector input signal, xᵢⱼ(kT+stₛ) is the sum of the measured output, yᵢⱼ(kT+stₛ) compensated by the detector's rise time response, the decayed outputs due to the input x-ray impinging at the detector at all previous viewing angles, 1, 2, . . . k−1, ie., xᵢⱼ(1), xᵢⱼ(2) xᵢⱼ(k−1).

It can be seen that the input signal impinging on the detector, or, xᵢⱼ(kT, stₛ) can be expressed as

xᵢⱼ(kT+stₛ)=yᵢⱼ(kT+stₛ)/a(1−e^{-st}^{ₛ}^{/γ}^{₁})−xᵢⱼ((k−1)T+stₛ)b₁e^{-st}^{ₛ}^{/τ}^{₁}−xᵢⱼ((k−2)T+stₛ)b₂e^{-(st}^{ₛ}^{+T/τ}^{₂}− . . . −xᵢⱼ(T+stₛ)bₖe^{-(st}^{ₛ}^{+(k-1)T)/τ}^{ₖ} (6)

Following scanning 92, detection 93, sampling 94, and conversion 95, the corrected sample 96 is then summed with the next acquired, corrected sample until the total viewing time is completed for imaging 98, i.e., the detector input signal at the end of the k^{th} view can be expressed as

$${x}_{ij} ( kT ) = {\sum }_{s = 1}^{S} {x}_{ij} ( ( k - 1 ) T + {st}_{s} ) \qquad ( 7 )$$

As can be seen from the above detector decay constant samples, a typical detector has time varying decay constants at the initial exposure of the x-ray flux, after several tenth ms, the detector reaches a final state of decay constant. At the k^{th} view, only the last (k−p)^{th} views have time constants that are time varying whereas all the previous (k−p−1)^{th}, . . . , k3, k2, k1 views have already reached the final steady state “afterglow” decay time constant. Let us define

Pᵢⱼ(pT)=xᵢⱼ(p−1)e^{-T/τ}^{ₚ}+xᵢⱼ(p−2)e^{-2T/τ}^{ₚ}− . . . −xᵢⱼ(1)e^{-(p-1)T/τ}^{ₚ}

It follows then

xᵢⱼ(kT+stₛ)=yᵢⱼ(kT+stₛ)/(1−e^{-st}^{ₛ}^{/γ}^{₁})−xᵢⱼ((k−1)T+stₛ)b₁e^{-st}^{ₛ}^{/τ}^{₁}−xᵢⱼ(((k−2)T+stₛ)b₂e^{-(T+st}^{ₛ}^{)/τ}₂− . . . −e^{-t}^{ₛ}^{/τ}^{ₚ}Pᵢⱼ(pT)

As stated before, the detector input signal at the end of the k^{th} view can be expressed as

$${x}_{ij} ( kT ) = {\sum }_{s = 1}^{S} {x}_{ij} ( ( k - 1 ) T + {st}_{s} )$$

In summary, the attenuated x-ray transmission flux through the object from given rotation angle k at the end of k^{th} view, xᵢⱼ(k), is the sum of all the sub-samplings during this period with the detector outputs properly compensated for both the detector rise time of the k^{th} view, also for the decay responses from all the previous k−1 views.

In the above, only a single detector response has been described. It follows that in a multi-slice CT system, there are xᵢⱼ(k) detector responses, where j=1, 2, . . . , J, represents number of slices of the CT scanner, and i=1, 2, . . . , I, represents number of detectors in a given row. Currently, there are single slice, double-slice, 4-slice, and up to 64-slice CT scanners in production, and there are 256-slice prototype systems.

A high-resolution, high-speed (i.e., >60 Mhz), bit-serial A/D is used in this implementation. The A/D outputs can be a series of 12 bits as opposed to having 12 parallel digital output bits. In this way the number of I/O pins and I/O communication wires of the signal processing boards are significantly reduced. Each serial digital output bits may be in the form of low voltage differential signaling (LVDS). The ADC with LVDS outputs have no difficulty in driving cables directly, but the quality of the cable determines the maximum frequency the cable can carry. The signal from the LVDS can be transmitted over 2 meter cable, for example

Note the following definitions unless indicated otherwise:

i=1, 2, . . . I, represents the number of channels within each row of the detector array.
Typical channel numbers are 800, 1000, i.e., generally in a range of 500 to 2000 channels;
j=1, 2, . . . J, represents the number of rows within each director array, or represents number of slices of the CT scanner, Typical slice numbers are 1, 4, 8, . . . 64, 128, 256;
k=1,2, . . . , K, represents the number of views of a given CT image, typical views are about 1000;
T=detector dwell time of a given view/orientation angle, typical view time is about 0.5 ms to 1 ms;
s=1,2, . . . 5, represents the number of sub sampled transmission data, typical total sub sample are 64, 128, 256;
tₛ=T/S, detector sub-sampling time within each view, where S represents the total subsampling integration number, Typical S are 64, 128, 256, etc;
tₘ=tₛ/M represents detector output multiplex time, where M represents the number of detectors time sharing a single A/D converter. Typical M is 4, 8, . . . , 32, 64, etc;
A Bit-serial output A/Ds are used to reduce the number of I/O cables. The available bit-serial A/Ds are list in Table 1.The digitized x-ray output data are corrected based on the detector rise-and-fall time characteristic and then all “S” sub-sampled data are digitally summed by the data processor onboard electronics to generate the output of a given view.

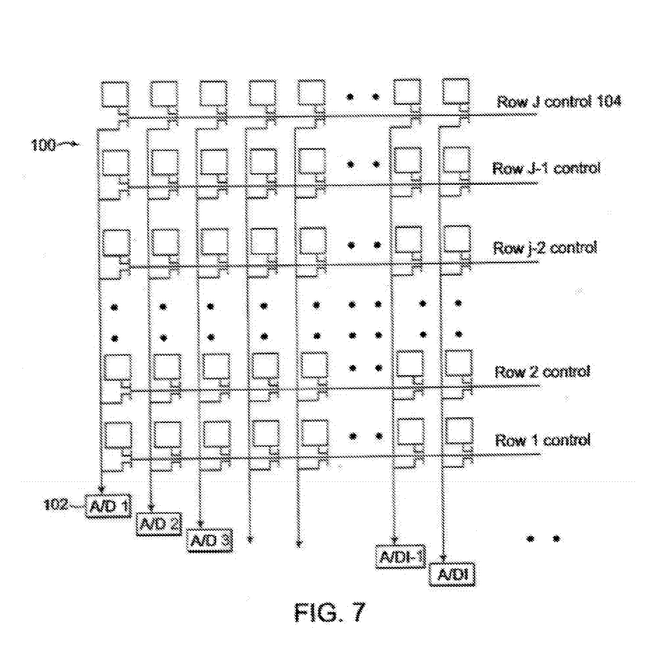

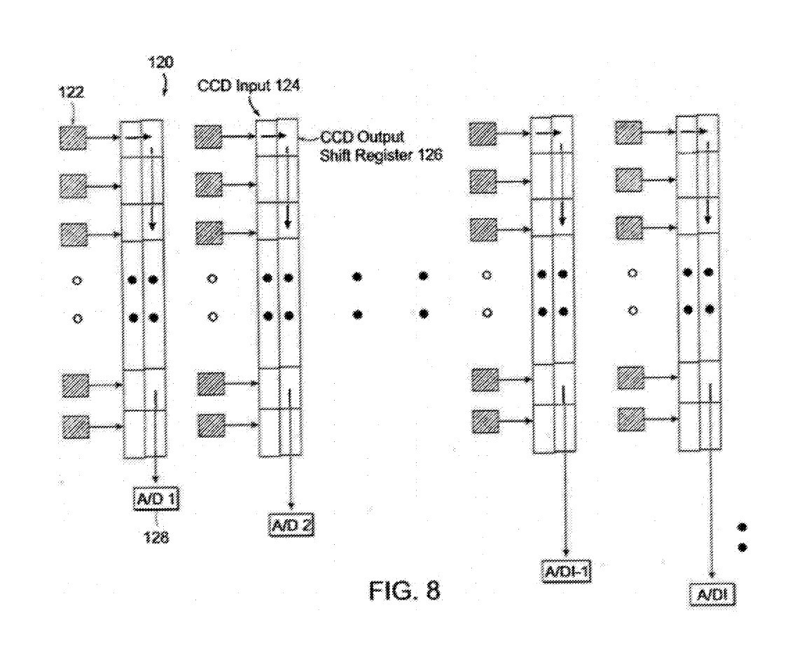

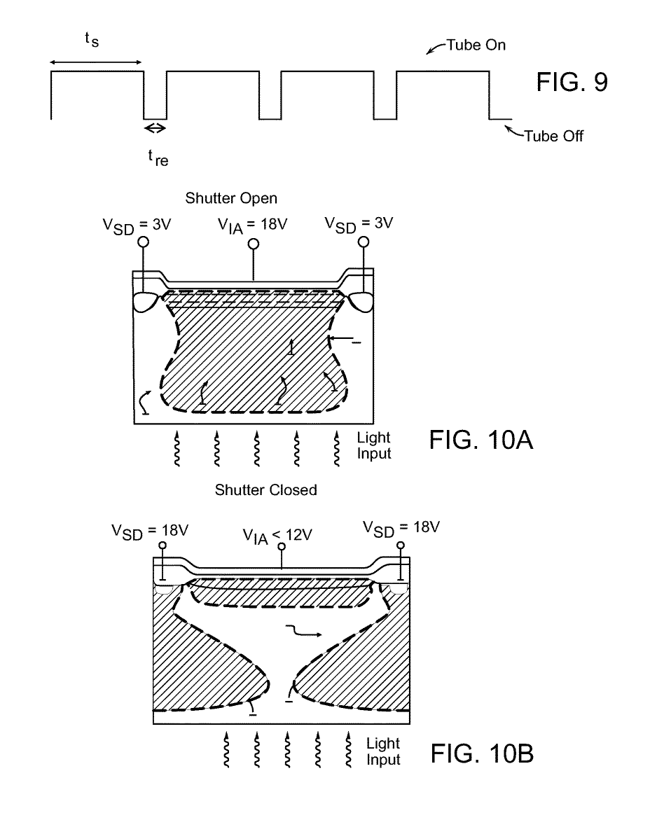

The time multiplexed detector output can be implemented within the detector array 100, as can be seen in FIG. 7, the output of each photodiode element is connected to a switching transistor. All the switch transistors on a given row 104 have a common control, i.e., for a detector array with J-row elements, there are J-control lines. Within each sub-sampling time, tₛ, a single impulse is clocked propagating through the J-control lines, which in turn allows the outputs of the photodiodes of each column be sequentially read-out at a clock rate of fₘ=1/tₘ, where tₘ=tₛ/M and M is the total number of photodiodes timing sharing a single A/D 102. In a CCD detector array 120 implementation, as shown in FIG. 8, charge from the photodiode array 122 of each column are parallel transferred 124 to a CCD parallel-in-serial-out output shift register 126 at the end of sub-sampling time tₛ, all the charges within the shift register are then serially clocked out at a clock rate of fₘ=1/t_{m.}

For the example of a CT scanner with a detector dwell time T be 0.5 ms. Let the total number of subsampling be 64,it follows then 1, be 7.8 us. Consider a 64-slice CT scanner, and let all the detectors along a given column share a single high-speed, bit-serial A/D. A single 60 MHz A/D is more than adequate to handle the entire detector along a given column. That is to say, for a 64 slice 1000 element CT scanner only 1000 A/D converters needs to be used, because all 64 elements in a given channel position can time-share the same A/D. For the above sample, the muxed detector output sampling rate, tₘ, is only 8 Mhz. As seen in Table 1, either Analog Device AD9222-50 or TI AS 5272 can be used for this application. Furthermore both A/D converters provide LVDS bit serial output. So, a 64-slice CT scanner with 1000 detector channels within each slice only needs 1000 pairs of LVDS digital outputs clocked at 96 MHz.

There are three basic physical mechanisms controlling charge transfer function in a charge domain processing device such as a CCD structure: self-induced drift, thermal diffusion and fringe-field drift. The self-induced drift dominates the beginning part of the charge transfer. Within a nanosecond, the effects of thermal diffusion and fringe fields determine the final charge transfer. In the following, the maximum clock rates for a digital imaging detector such as a CCD under the thermal diffusion limitation and under the fringe field limitation are computed, it is clearly demonstrated that for a large area CCD, with a thousand charge transfers, greater than a 100 Mhz clock rate is achievable with a buried channel fringe field implementation.

The time constant of thermal diffusion can be expressed as follow, where D is the electron diffusivity.

$$\begin{matrix}{τ}_{th} = \frac{4 {L}^{2}}{{π}^{2} {D}_{n}} \\ t = ( - {τ}_{th} ) ln ( \frac{ɛ ( 1 + \frac{{Q}_{o}}{{Q}_{th}} )}{1 + ɛ \frac{{Q}_{o}}{{Q}_{th}}} ) \\ where \\ {τ}_{th} = \frac{4 {L}^{2}}{{π}^{2} {D}_{n}} {Q}_{o} = 4 CL {Q}_{th} = 2 \frac{kT}{q} CL \\ \begin{matrix}L & {τ}_{th} & t ( ɛ = {10}^{- 5} ) & \begin{matrix}{f}_{c} \\ 2 phase clock\end{matrix} \\ 3 μ m & 1.76 ns & 12.6 ns & 39 MHz \\ 5 μ m & 488 ns & 35 ns & 14 MHz \\ 8 μ m & 12.5 ns & 89.5 ns & 5.6 MHz\end{matrix}\end{matrix}$$

As can be seen above, under the thermal diffusion limitation for a surface channel CCD, the maximum clock rate of an 8 um gate CCD is only about 5.6 Mhz.

The time constant of the fringe field is derived as follows: instead of thermal diffusivity, the fringe field generates an additional field induced diffusivity that drastically improves the electron mobility and enables less than 0.00001 transfer inefficiencies. A buried channel CCD structure can be used to generate the fringe field. In this structure, the signal charge is confined in the N-implanted layer away from the surface in a region in which the electric fringe field between transfer gates is increased, preferably to a maximum. The fringe electric field is determined by the geometry, material parameters and external clock voltage and may be approximated by the expression:

$${E}_{fr} ( min ) = \frac{Δ V}{L} ( exp ( - \frac{π {k}_{s} {ɛ}_{o}}{3 {C}_{eff} L} ) - exp ( \frac{π {k}_{s} {ɛ}_{o}}{{C}_{eff} L} ) )$$

where C_{eff} is defined by the expression

$$\frac{1}{{c}_{eff}} = \frac{{x}_{o} ( eff )}{{k}_{o} {ɛ}_{o}} + \frac{d}{{k}_{s} {ɛ}_{o}} ( 1 - \frac{Δ d}{d} - \frac{{Q}_{s}}{2 {N}_{ion}} ) .$$

Where Xₒ is the effective oxide thickness, d is the distance from the Sᵢ/SᵢO₂ interface into the silicon, where the centroid of the charge is located, delta d is the width of the charge centroid, Qₛ is the signal charge density, and Nᵢₒₙ is the effective donor concentration in the epitaxial or ion-implanted region.

Where delta V is the voltage difference between the transfer electrode and the receiving electrode (L is the length of the transfer electrode). The minimum fringe field is important because, for small channel lengths, the remaining charge is confined to the center of the gate electrode where the fringe field is a minimum. The effect of the fringe field can be included in the field enhanced diffusion constant which may be written as

$${D}_{eff} = D + \frac{2 μL}{π} {E}_{fr} ( min ) .$$

Therefore, the time constant of charge transfer in the final stage is given as

$${τ}_{eff} = \frac{4 {L}^{2}}{{π}^{2} {D}_{eff}} .$$

The transfer inefficiency can be written as

ε(t)=exp(−t/τ_{eff})

Where, in a two phase clock structure, the time allotted for charge transfer is given by

t(max)=(½)f_{c}

Where f_{c} is the clock frequency.
It follows then

$${f}_{c} = \frac{{π}^{2} μ}{8 {L}^{2} ln ( 1 \text{/} ɛ )} ( \frac{kT}{q} + \frac{2 L}{π} {E}_{fr} ( min ) )$$

Using, for example, the design parameters:

ΔV=4V

E=10⁻⁵

x_{O}=1.28×10⁻⁶ cm

d=0.75×10⁻⁴ cm

Table 2 Maximum Clock rate as a function of Fringe field and gate length

| Gate Length | E_{fr} (min) | f_{c,} Maxiumum Clock Rate |
|---|---|---|
| L = | V/cm | Mhz |
| 3 um | 12,847 | 3,057 |
| 5 um | 3,685 | 526 |
| 8 um | 1,256 | 112 |

With the fringing field, an 8 um gate buried channel CCD (BCCD) can easily reach greater than a 110 Mhz clock rate.

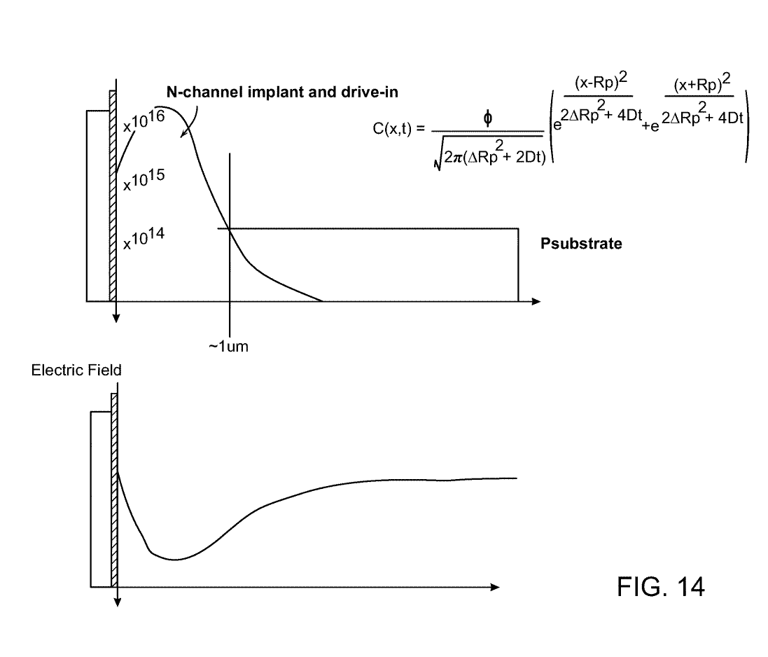

The starting material of a preferred BCCD is a high-resistivity p-type Si wafer with doping concentration around 10¹⁴ cm⁻³ range. Either a bulk silicon (Si) wafer or high-resistivity epi wafer can be used. The buried channel is formed by phosphorus or arsenic implantation followed by high temperature thermal drive in of the implant (FIG. 14). After implantation, the wafer is exposed to a series of high-temperature processing cycles, at temperature T₁, time duration t₁, temperature T₂, time duration t₂, etc. The corresponding diffusion constant is D₁, D2, . . . , respectively. Let us define DT=D₁t₁+D₂t₂+D₃t₃+ . . . , as the sum of Dt products is sometimes referred to as “thermal budget” of the process. The N channel distribution, C(x,t), is defined by the following equation.

$$C ( x , t ) = \frac{Q}{\sqrt{2 π ( Δ {Rp}^{2} + 2 Dt )}} ( {e}^{- \frac{{( x - Rp )}^{2}}{2 Δ {Rp}^{2} + 4 Dt}} + {e}^{- \frac{{( x + Rp )}^{2}}{2 Δ {Rp}^{2} + 4 Dt}} ) .$$

Where Q is the implant dose per cm², Rₚ, delta Rₚ are implant range and implant struggling values associated with the implant energy; respectively.

For a preferred BCCD, the implant dopant dose, energy and thermal budget are carefully selected such that the imager can be operated at a high frame/clock rate.

The duration of the exposure, exposure time, in current x-ray systems is in the range of 0.5 s to 1.0 s, depending on the equipment. During the exposure time, T, each pixel of the detector array is integrating the instantaneous incident optical photons generated by the scintillator. In the preferred embodiment, during the exposure time, instead of integrating continuously in each pixel, after an exposure of tₛ each frame is quickly ready in a time, tᵣₑ, and the subframes are summed digitally to provide the final images.

$${I}_{ij} ( T ) = {\int }_{0}^{T} {I}_{ij} ( t ) dt = {\sum }_{n = 1}^{N} {\int }_{( n - 1 ) ( {t}_{s} + {t}_{re} )}^{( {n t}_{s} + ( n - 1 ) {t}_{re} )} {I}_{ij} ( t ) dt .$$

The time in this sub exposure and read-out approach is T+N tᵣₑ. It is important to note that this digital integration is correct, only if the tᵣₑ is fast enough that the target movement can be ignored during this additional read-out time. In this embodiment, the tᵣₑ is in the range of 100 ms or less. Another important parameter is that the x-ray source is to be turned off during the read-out time.

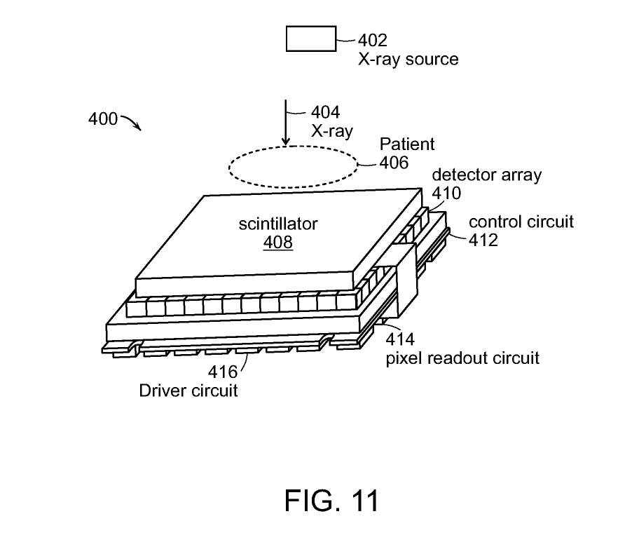

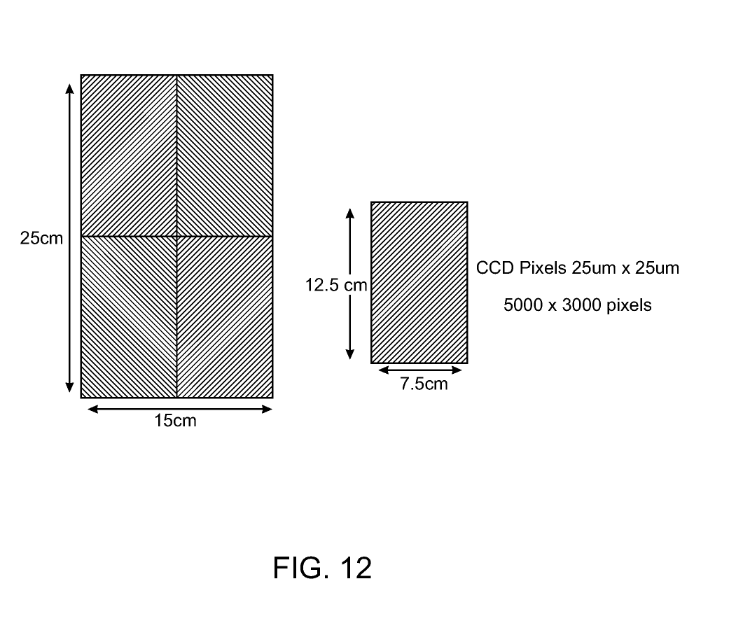

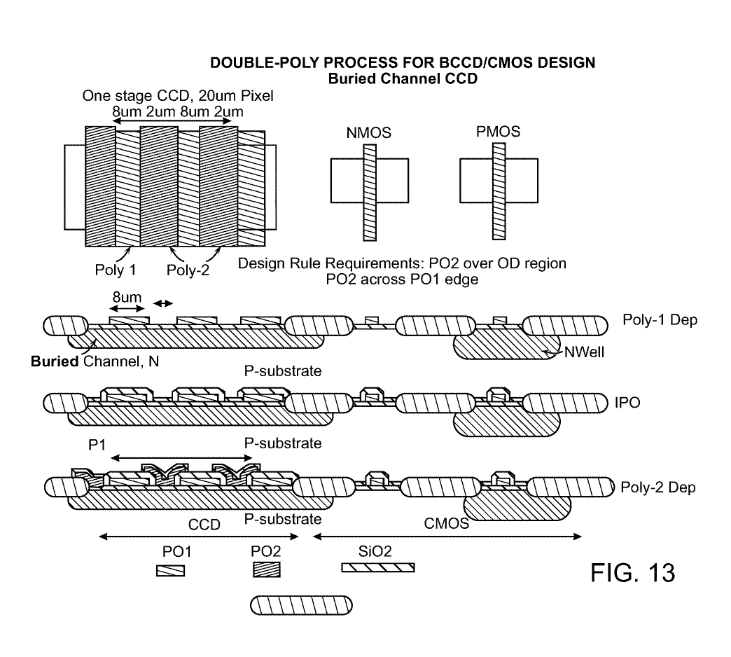

A back-illuminated CCD 410 has been developed to greatly improve sensitivity over commercial imagers by bringing light into the device through the back surface, unobstructed by structures on the front surface. FIG. 11 represents a view of the physical structures in the silicon detector device 400. The device includes a scintillator 408 coupled to the array that is controlled by driver 416 and read-out circuits 414. A digital circuit board 412 transmits the images for external processing and storage. One feature not found in a typical CCD is the p+ buried layer (see FIG. 13), implanted with a high-voltage implanter (about 1 MeV boron). This layer forms a potential barrier that separates the illuminated back surface (bottom) from the front surface (top) of the device. Thus, the buried layer provides a barrier layer to reduce or eliminate signal interference.

For a dynamic x-ray imaging system, high speed shuttering is needed to eliminate image blur caused by continuous incoming incident signal while the detector array is in a read-out mode. In current phased array radars for example, typical radar transmitter requires tens or hundreds voltage transmitter with KW power in sub microseconds. Diversified Technology's solid-state switches operate in as little as 50 nanoseconds. Switches require only 110 VAC power for operation. For example, 55 kV 50 A switch module assembly that modulates the TWTs in the Cobra Judy s-band transmitter system has been produced by Diversified Technology.

A high speed switch that can turn off a high-voltage power supplier in micro seconds can be incorporated in the present system. Alternatively, an electronic shutter that can be integrated into the detector design.

FIG. 10A represents the depletion region of the device storage well (blue region) when the storage well gate (V_{IA}) is moved to a very high potential (18 V). The depletion region has reached through the p+ buried layer, and so photoelectrons (represented by the—symbols) may move from the back surface of the device, where they are generated, to the storage well under V_{IA} on the CCD front side. This is the “shutter open” condition.

FIG. 10B represents the potential configuration of the device when the storage well gate is moved to an intermediate voltage (<12 V), which is high enough to maintain a storage well (and the charge in it) under the gate (blue region), but not high enough for the depletion region to reach through the deep implant.

The shutter function also uses shutter drains running down the channel-stops as two n+ diodes imbedded in the p+ channel stop region and connected to a voltage V_{SD}. While the storage-well voltage is reduced, the shutter-drain voltages are increased, allowing their depletion regions (shaded) in the shutter closed diagram (FIG. 10B) to now reach through the deep buried layer and form a way for the photoelectrons to be drained out of the pixel and discarded. Use of the shutter drain prevents photocharge generated during the shutter-closed condition from building up to the point of leaking over the potential barrier and contaminating previously collected charge in the CCD well.

The electronic shutter structure has enabled a number of devices, such as a four-sample high-speed burst imager and a fifty-sample imaging device, by making it possible to shelter previously collected charge from current photocharge when the integration period of the imager has ended. Both of these devices operate with effective sampling rates well above 1 MHz. Once the photon collection event is over, the multiple stored images are read-out with low noise at manageable data transfer speeds so the noise is low.

The BCCD array can be clocked at greater than 100 v Mhz clock rate. With 5 parallel read-out ports, this allows a large array with 4000 by 4000 side elements to be read-out at a 30 ms frame rate. With 10 parallel read-out ports, it only takes 16 ms to read-out a whole 4000×4000 element frame. This high-frame rate read-out allows the detector array operating both in continuous visualization (fluoroscopic) and in static imaging applications. However, for real-time visualization fluoroscopic mode, it is important to eliminate image blur while the image is frame-transfer mode. A preferred solution is combining a back-side illumination detector array with a high-speed electronic shutter, the shutter can be turned on while the previously acquired frame is being read-out.

It is desirable to provide a CCD imaging system using a high-speed electronic shutter that can be specifically incorporated into the structure of the CCD, e.g., of a back-illuminated, frame-transfer CCD, to provide a device having a simpler structure that can be produced at reasonable costs. Such a device is arranged to operate in a manner such that smear is substantially reduced, or eliminated, a high pixel fill factor (substantially at, or close to, 100%) is achieved, flexible integration times are made available, low-noise operation occurs, and near-reflection-limited quantum efficiencies over the visible spectrum can be simultaneously obtained. While other solutions to the problem of image smear, when the integration time is comparable to or less than the transfer time, have been proposed, none has been able to achieve such desired overall operation. For example, it has been suggested that image smear can be removed by performing post-processor data operations on the image data using suitably designed algorithms Such an approach, however, is accomplished at the expense of time and increased hardware cost.

A CCD imaging system that can be operated in a back-illuminated mode with a high-speed electronic shutter structure that is integrated during the CCD fabrication process with a simple additional implant mask.

In some embodiments, the scintillator can include cesium iodide or gadolinium oxide sulfur. The cesium iodide can be thallium-activated to improve, for example, luminous efficiency. In accordance with various embodiments, the scintillator can include columnar crystals of a material wherein the x-ray radiation is converted to visible light and is emitted from a distal end of the columns. The columns can have a shape that promotes light-guiding of the emitted light. The distal ends of the columns may be formed with a shape such as a taper having an angle to modify light extraction capabilities, and the distal ends may be covered by a protective film to reduce moisture penetration and/or abrasion. The scintillator may be grown at least in part using vapor deposition or evaporation techniques.

Another feature is the scintillation and CCD arrays can be mounted on a flexible plastic substrate(s), such as polyimide, polyether ether ketone (PEEK) or transparent conductive polyester film, the technique is known as flexible electronics or flex circuits.

X-rays are generated by an x-ray source which passes through a region of a subject's body, forming an x-ray image which reaches a scintillation screen (e.g. sodium iodide) and then to a detector array. An x-ray source such as a carbon nanotube source, such as that described by Shan et al, “Stationary chest tomosynthesis using a carbon nanotube x-ray device array: a feasibility study”, Phys. Med. Biol. 60 (2015) 81-100 and in U.S. Pat. Nos. 8,995,608, 8,600,003, 8,358,739 and 9,167,677 (the entire contents of the publication and patents being incorporated herein by reference) can be used in preferred embodiments. Such a carbon nanotube source can comprise an array of carbon nanotube emitters that emit an electron beam. In various embodiments, the x-ray source can be stationary or moving with respect to the object to be imaged. The x-ray source can be spatially distributed and can include an array of individually activated x-ray source focal spots. In some embodiments, the array of x-ray focal sources can include 75 or more sources. In some embodiments, each focal spot emanating from the array of x-ray source focal spots can be less than 1×1 mm in size. The x-ray source can include a collimating element that can adjust the output beam width. This control of the beam width can prevent or limit the irradiation of objects that the user does not wish to image such as normal tissue in a patient.

The scintillation layer emits an intensity pattern corresponding to the x-ray image, and then image then detected by a detector array. It can be seen in FIG. 1, a conventional flat rigid-detector array with a scintillator replaced with an curved array, where flexibility is provided through the use of polyimide (PI) as a substrate, for example.

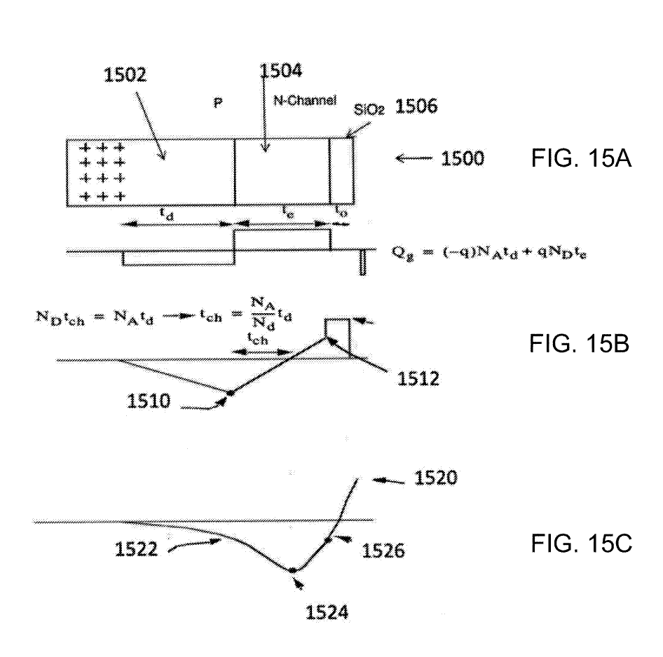

An example of a device 1500 in relation to the present invention is shown in FIG. 15A. The device 1500 includes a p-type channel 1502 having a depletion region of width t_{d} and an n-type channel 1504 having a depletion region of width tₑ, and an insulating silicon dioxide layer of width tₒ. The total charge in the device 1500 can be expressed as Q_{g}=(−q)N_{A}t_{d}+qN_{D}tₑ. A representation of the charge distribution for the device 1500 in certain embodiments is shown in FIG. 15B. At a position 1510 on the p-n junction, the charge can be given as qN_{A}t_{d}/κₛεₒ. After rising through the n-channel 1504, a position 1512 on the n-channel/insulator junction can be given as q(N_{D}tₑ−N_{A}t_{d})/κₛεₒ. In FIG. 15C, an energy diagram 1520 is shown for certain embodiments of the device 1500. The potential can be described as V_{g}=φₛ+q(N_{A}t_{d}−N_{D}tₑ)tₒ/κₛεₒ. The long tail 1522 of the curve to the left in the figure can be described as qN_{A}t_{d}²/2κₛεₒ. The bottom of the potential well 1524 can be described as qN_{A}(N_{A}+N_{D})t_{d}²/2κₛεₒN_{D}=φ_{ch}. The curve rises out of the well to position 1526 that can be described as where φₛ=q(N_{A}t_{d}²+tₑ(2N_{A}t_{d}−N−_{D}tₑ))/2κₛεₒ.

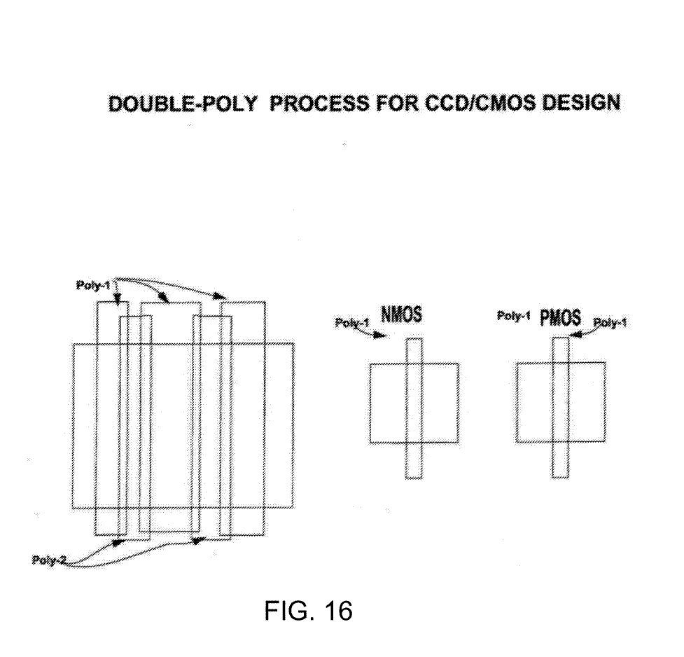

FIG. 16 illustrates an overview of a double-poly process for CCD/CMOS 1600 design in accordance with various embodiments of the present invention. The NMOS element 1602 utilizes a first polysilicongate and the PMOS element 1604 utilizes a second polysilicon gate.

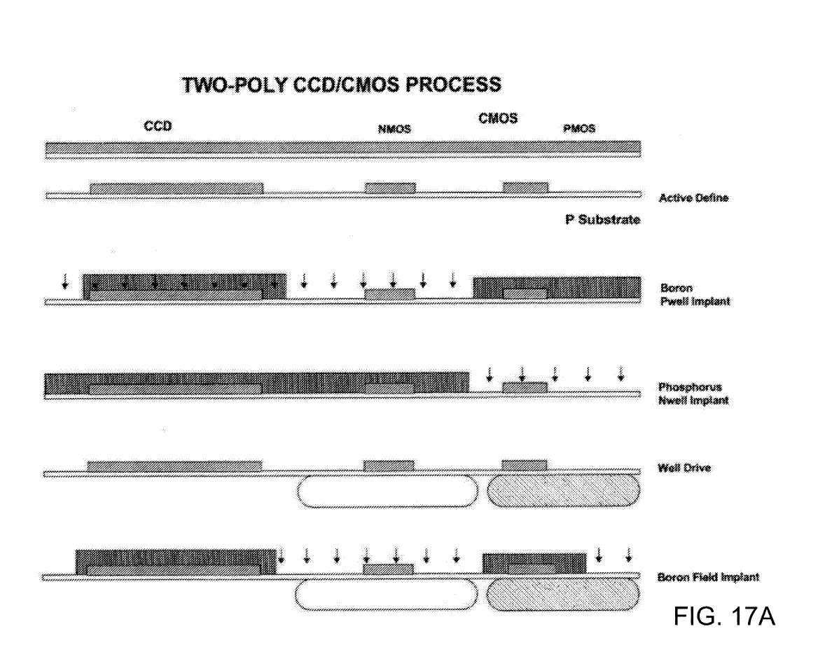

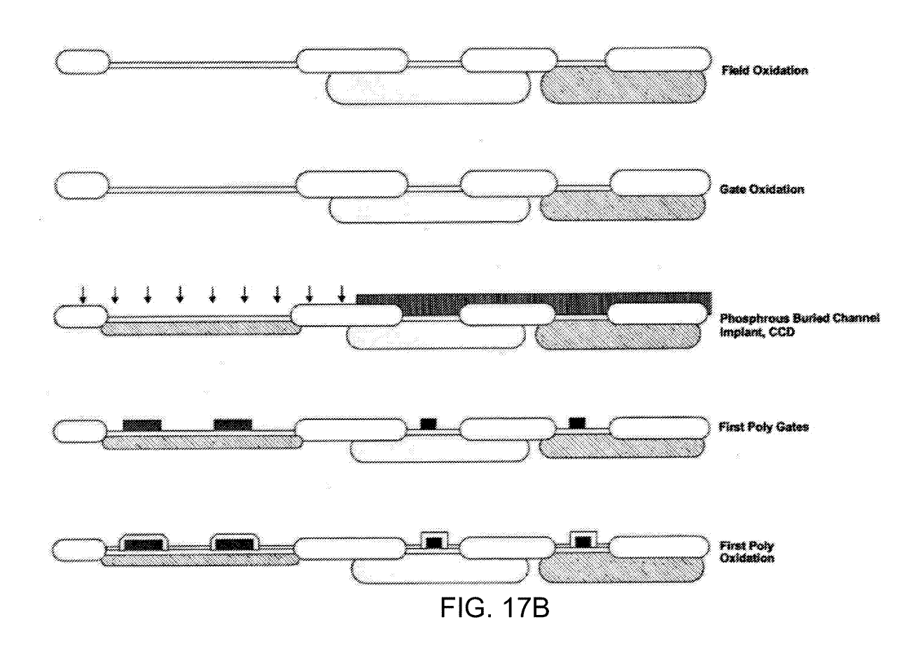

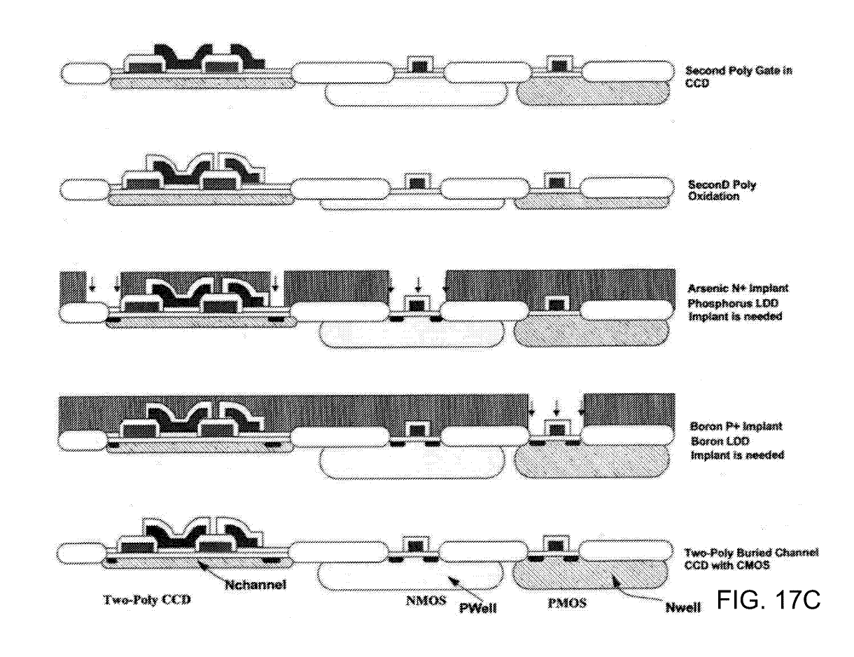

FIGS. 17A-C schematically illustrate steps of a two-poly CCD/CMOS process 1700 in accordance with the invention. In step 1702, a wafer is provided containing sections suitable to form the CCD and CMOS portions. Step 1704 is an active define step and p-well implantation such as with boron occurs in step 1706. An n-well implant such as with phosphorus can occur in step 1708. Step 1710 can include well drive while step 1712 can include boron field implant. Steps 1714 and 1716 can include field and gate oxidation, respectively. In step 1718, a buried channel implant can occur such as with phosphorus in the region of the CCD. Steps 1720 and 1722 can include formation of the first poly gates and the first poly oxidation, respectively. In step 1724, a second poly gate can be formed in the vicinity of the CCD. A second poly oxidation can be included in step 1726. In step 1728, an arsenic n+ implant can occur and a phosphorus LDD implant is needed. In step 1730, a boron p+ implant can occur and a boron LDD implant is needed. Step 1732 is depicted in FIG. 17C as including the full two-poly buried channel CCD with CMOS.

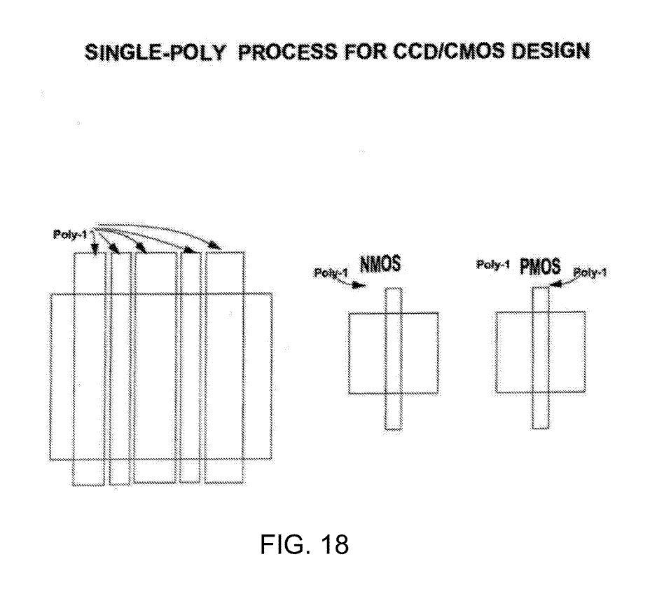

FIG. 18 illustrates an overview of a single-poly process for CCD/CMOS design 1800 in accordance with various embodiments of the present invention. In this design, the gate structure 1802, 1804 is used for both NMOS and PMOS elements.

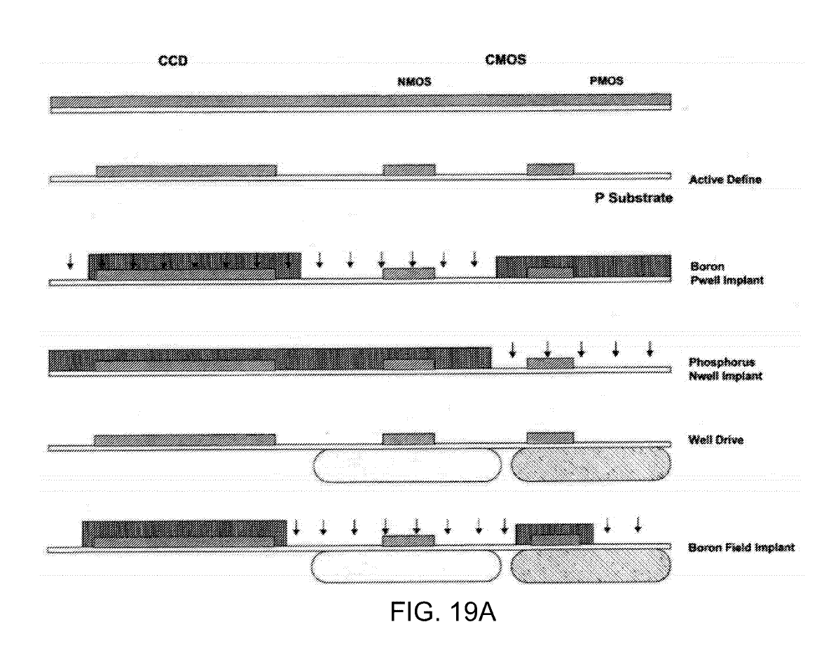

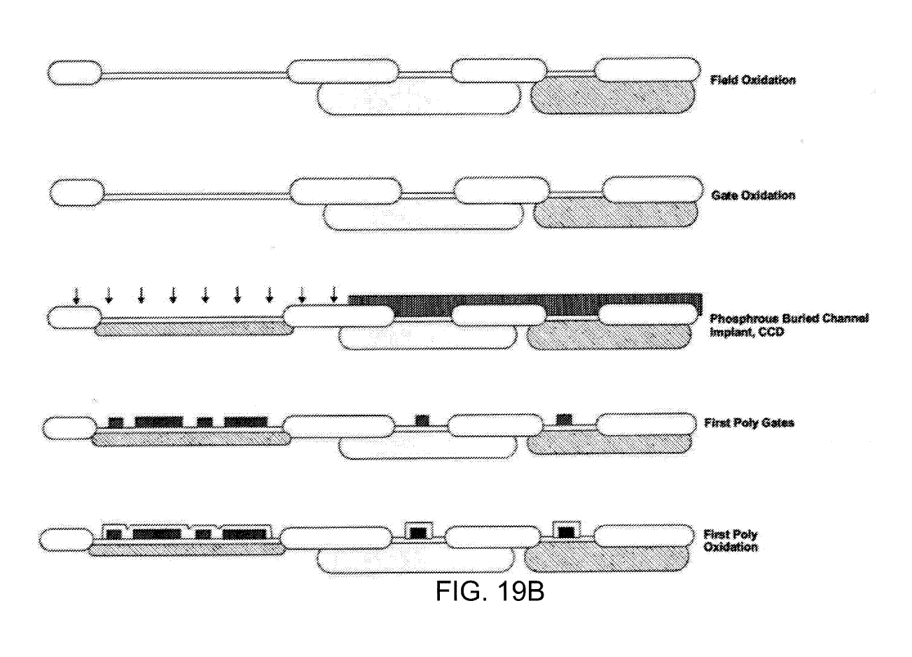

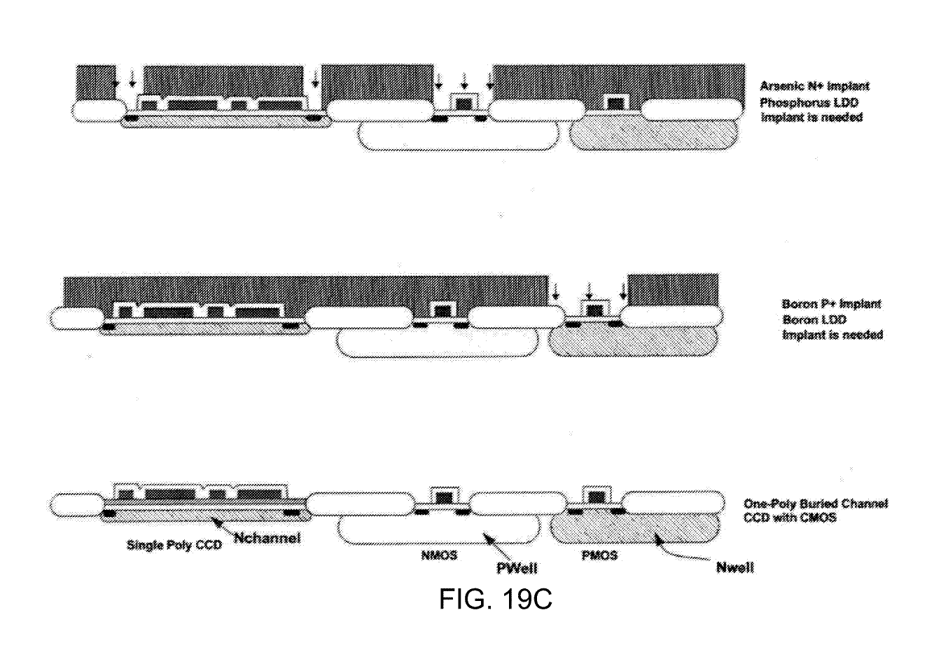

FIGS. 19A-C schematically illustrate steps of a one-poly CCD/CMOS process 1900 in accordance with the invention. In step 1902, a wafer is provided containing sections suitable to form the CCD and CMOS portions. Step 1904 is an active define step and p-well implantation such as with boron occurs in step 1906. An n-well implant such as with phosphorus can occur in step 1908. Step 1910 can include well drive formation while step 1912 can include boron field implant. Steps 1914 and 1916 can include field and gate oxidation, respectively. In step 1918, a buried channel implant can occur such as with phosphorus in the region of the CCD. Steps 1920 and 1922 can include formation of the first poly gates and the first poly oxidation, respectively. In step 1928, an arsenic n+ implant can occur and a phosphorus LDD implant is needed. In step 1930, a boron p+ implant can occur and a boron LDD implant is needed. Step 1932 is depicted in FIG. 19C as including the full one-poly buried channel CCD with CMOS.

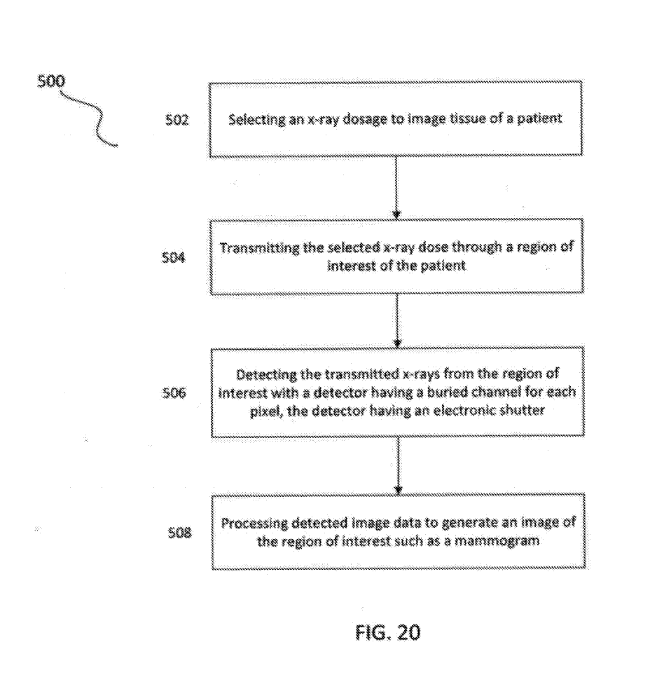

Shown in FIG. 20 is a method of imaging 500 a region of interest in a patient with devices and methods described herein. A dosage is selected 502 based on the type of study to be conducted and the characteristics of the patient. The dosage can be automatically selected based on programmed “pre-sets”. The x-ray scan or imaging sequence is performed 504 and an x-ray detector as described herein is used to detect the x-rays 506. The detected image data is then processed 508 to form one or more images of the region of interest.

The above process sequences enable formation of a buried channel integrated circuit device used to form an x-ray detector for medical imaging applications. The detectors described herein can be used in conjunction with the x-ray sources set forth above to provide a back illuminated detection system using a scintillator to convert the incident x-ray energy transmitted through the patient into an optical signal for detection. This system provides a pixel size of less then 75 microns and preferably less then 50 microns to provide improved resolution and decrease the required x-ray dose.

The claims should not be read as limited to the recited order or elements unless states to the effect. All embodiments that come within the scope and spirit of the following claims and equivalents thereof are claimed as the invention

What is claimed is:

1. An x-ray detector system comprising:
a detector array having a plurality of pixel elements, each pixel element having a buried channel; and
a scintillator coupled to the detector array.

2. The system of claim 1 further comprising an electronic shutter.

3. The system of claim 2 wherein the electronic shutter comprises a depletion region at each pixel element.

4. The system of claim 1 wherein the detector array is back illuminated.

5. The system of claim 1 further comprising a flexible substrate wherein the detector array is mounted to the flexible substrate.

6. The system of claim 1 further comprising an x-ray source.

7. The system of claim 6 wherein the x-ray source comprises a carbon nanotube source.

8. The system of claim 6 wherein the x-ray source comprises a solid state source.

9. The system of claim 1 wherein the detector array comprises a charge couple device wherein the buried channel generates a fringe field.

10. The system of claim 1 wherein the scintillator comprises a flat or curved element mounted on a silicon detector array device.

11. The system of claim 1 wherein the detector comprises a CMOS device.

12. A method of imaging a region of interest comprising:
transmitting x-rays from an x-ray source through a region of interest;
converting transmitted x-ray to an optical signal with a scintillator; and
detecting the optical signal with a detector device having a plurality of pixel elements, each pixel element having a buried channel region.

13. The method of claim 12 further comprising forming a mammographic image of tissue of a patient.

14. The method of claim 12 further comprising forming a plurality of CT images.
